(19) United States
(12) Patent Application Publication
(10) Pub. No.: US 2019/0156952 A1
(43) Pub. Date: May 23, 2019

(54) Adjustable socio-economic indexing system

(71) Applicant: KONINKLIJKE PHILIPS N.V., Eindhoven (NL)

(72) Inventors: Eran SIMHON, Boston, MA (US); Xin WANG, Cambridge, MA (US); Reza SHARIFI SEDEH, Malden, MA (US); Amir ABDOLAHI, Waltham, MA (US); Cecilia MEIJER, Cambridge, MA (US)

(21) Appl. No.: 16/174,336

(22) Filed: Oct. 30, 2018

Related U.S. Application Data
(60) Provisional application No. 62/587,921, filed on Nov. 17, 2017.

(51) Int. Cl. G16H 50/30; G16H 50/50; G06N 99/00

(52) U.S. Cl. CPC G16H 50/30; G06N 99/005; G16H 50/50

(57) ABSTRACT

In one embodiment, a computer system and method is disclosed for providing an adjustable social determinates of health model, which allows for user selectable customization of the categories used to calculate the model as well as adjustment of the model itself. In addition, the system displays predictiveness metrics indicating the predictive quality of the model. The model can be used by the user to create a socio-economic index to provide the user with insights into the relevant socio-economic factors impacting healthcare of an individual patient or population.

### CROSS-REFERENCE TO PRIOR APPLICATIONS

This application claims the benefit of U.S. Patent Application No. 62/587,921, filed on 17 Nov. 2017. This application is hereby incorporated by reference herein.

### FIELD OF THE INVENTION

The present invention is generally related to computer systems for providing health measures, and more particularly, to computer systems for providing a patient health index which takes into account socio-economic determinates of health.

### BACKGROUND OF THE INVENTION

The delivery of healthcare is evolving rapidly driven in no small part by the high cost of health care and an aging worldwide demographic with increased healthcare needs. Without systemic changes, there will be an increased percentage of the population without meaningful access to healthcare. Historically, patient care was performed on a case-by-case basis focused on episodic interventions by a care provider often at the request of the patient in a fee-for-service relationship.

In addition, care providers may be generalists, or specialists with a particular field of expertise, and may lack expertise in every possible medical nuance of a given patient's total clinical condition. Therefore, these care providers may only take into account a subset of the issues the patient is struggling with based on their particular expertise and miss other important issues. This may result in the caregiver failing to address some critical health issues. Or even worse, in some instances, their guidance may even create, or exacerbate, the other health issues.

Further, caregivers often do not have immediate access to the patient's complete medical history. Patient records are often unavailable, incomplete, or the caregiver may simply not have sufficient time to review the records in the time allotted. Care providers often have to rely on their personal judgment to make the best care decision possible with limited information and limited time allotted during a private consultation. Such decisions could end up being biased, outdated, based on erroneous information or otherwise suboptimal.

Due to the issues noted above, this method of patient-initiated, episodic care management has been found to have several drawbacks. Patients left to manage their own care tend to postpone seeking medical counsel until late stages in their disease progression, thus increasing healthcare costs with a resulting decreased quality of life. This is in contrast with early interventions, which are known to decrease or eliminate negative health consequences at reduced cost.

In the fee-for-service system, the caregiver may have the wrong incentive. Since they are compensated based on services rendered and penalized for poor outcomes. Their financial motivation is aligned with maximizing the delivery of services, which may be at odds with the cost-effective delivery of care. And, if they happen to overlook something they could be held responsible so it is better to over prescribe diagnostics and therapy, even if the likely need is remote. And, in many instances, once the patient seeks medical counsel, they too are often unable or unmotivated to evaluate the counsel given. They are handicapped by their lack of medical expertise, and when they are covered by health insurance, they too have little motivation to control costs. So, when the care provider stands to profit and the patient has little or no ability to disagree, expensive interventions with remote therapeutic value may be pursued thus burdening the healthcare system and needlessly raising costs.

To address the inefficiency of fee-for-service healthcare, government and private payers have sought to correct this situation and provide compensation to caregivers based on their performance rewarding superior outcomes and cost-efficiency otherwise known as value based care. While theoretically desirable, it has proven difficult to develop metrics, which create the right incentives. If the focus is solely on outcomes, then any intervention even with modest therapeutic benefit should be pursued. The obvious down side to this approach is, as with episodic care, this method will result in waste and extreme cost. On the other end of the spectrum, there an incentive system could focus solely on costs. Taken to an extreme, cost savings can be realized by refusing even therapeutic service leading to poor outcomes. So, as there has been a migration from a fee-for-service model to these incentive-based systems, it has pitted patients and physicians against payers. Patients and physicians often accuse payers of putting profits ahead of delivering on their promise of providing health coverage. And, payers argue that patients seek costly interventions when the therapeutic value is suspect. Intellectually, the problem becomes one of optimization, the goal should be to optimize care to provide the highest quality at the lowest cost. However, given the issues noted above, identifying the optimized care has proven challenging especially when the goal is to develop an intervention for a particular patient with unique characteristics.

In an effort to address this problem, there has been a rise in evidence-based medicine to support the shift from the fee-for-service model to value based care. Rather than relying on the expertise of a singular caregiver, evidence-based medicine applies scientific principles to the delivery of care in order to optimize decision-making based on a review of longitudinal patient data to develop standards of care or best practices. In these models, metrics are developed to compensate the caregiver based on their performance and seeks to reward superior results relative to their peers.

While providing clear advantages, one challenge with evidence-based medicine has been the collection, storage, and analysis of large volumes of patient data in an effort to provide a customized care plan for patients or populations. To mitigate this issue, electronic medical records systems (EMRs) have been developed to assist with data management. These systems typically include the same information that would have been retained by individual caregivers across the healthcare network. Advantageously, EMRs provide a centralized repository for patient records including patient history, demographics, past visits, imaging, and the like. Collecting these records in a central repository facilitates caregiver collaboration and the longitudinal data can be used for better evidence-based care analytics thus providing a fuller picture of the patient's clinical picture.

While the shift to evidence-based medicine and analysis of EMR data has been an improvement, additional advances are desired. Disappointingly, it has been found that demographic and healthcare factors typically captured in EMRs are not as predicative of the patient's healthcare outcomes as one would expect and are, in fact, somewhat poor at predicting a patient's health status and likely outcome, thereby undermining the promise of evidence-based care as a means to achieve optimized healthcare. It has been found that equally, if not more predicative of a patient's health status and outcomes, are socio-economic factors such as economic, environmental, educational, social, and behavior factors. The data contained in current EMRs provides an imperfect picture of the patient and lacks any insight into these important socio-economic determinates of health. Moreover, as payers have shifted to a payment system that rewards efficiency, there is understandable focus regarding how efficiency is calculated. It is not uncommon for caregivers identified as low performers to argue that their low score isn't attributable to their inefficiency, but instead is due to the fact that the population they serve is more complicated than the average thus leading to worse outcomes or need more costly interventions. While there is some acceptance that no caregiver has a representative patient cross-section, it is hard to validate these assertions since the current tools available do a poor job at capturing these socio-economic determinates of health or evaluate how they impact outcomes or cost. The present invention seeks to overcome one or more of the deficiencies noted above.

### SUMMARY OF THE INVENTION

One object of the present invention is to provide a flexible socio-economic model system. The system includes a computing device to performing computing functions. The system also includes at least one database including socio-economic data. A user device is provided, which has a graphical interface presented on a display. The graphical user interface displays socio-economic categories. The user may select which socio-economic categories should be used in the calculation. Next, the computing device then trains a model using machine learning and provides on the graphical user interface the calculated model that correlates one or more socio-economic categories with a socio-economic score as well as at least one predictiveness metric indicating the predictive quality of the model. The socio-economic model includes socio-economic factors selected from the selected socio-economic categories which are related by weighting factors. The predictiveness metrics indicate the quality of the socio-economic model to correlate the selected socio-economic categories with the socio-economic score.

In another embodiment, the present invention provides a computer-implemented socio-economic indexing system. The system includes receiving individual data, electronic medical records data, and socio-economic data about individuals. The system further allows a user to select socio-economic categories to be used by the computer to calculate a socio-economic score. Once the selections have been made the system performs an analysis on the received data and creates a model which includes socio-economic factors and weighting factors. The weighting factors and socio-economic factors are correlated to the socio-economic score. The model calculated by the system is then displayed on the graphical user interface of the user device as well as at least one predictiveness metrics. The predictiveness metric allows the user to evaluate the quality of the model generated by the computer-implemented socio-economic modeling system.

In another embodiment, the present invention provides a non-transitory computer readable medium with executable instruction that when executed by one or more processors causes the processors to receive individual data, electronic medical records data, and socio-economic data about individuals. The processers are programmed to cause the graphical user interface of a user device to display socio-economic categories. The display permits the user to select the socio-economic categories for use in calculating the socio-economic model. Once the socio-economic categories are selected various factors are subsequently selected. The processors then train a socio-economic model, which correlates the factors with the socio-economic score. This correlation occurs via a plurality of weighting factors. Together the socio-economic factors and their associated weighting factors define the socio-economic score. The processors then render the socio-economic model and one or more predictiveness metrics on the graphical user interface of the user device.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a schematic diagram of an example network environment in which an example socio-economic index system, in accordance with an embodiment of the invention.

FIG. 2 is diagram of socio-economic categories and factors, which may be used when generating an socio-economic model.

FIG. 3 is an embodiment of a data structure of socio-economic categories and select socio-economic factors in accordance with an embodiment of the present invention.

FIG. 4 is a flow diagram of an example process for generating a socio-economic model in accordance with an embodiment of the invention.

FIG. 5 is a schematic diagram that illustrates the components of a socio-economic score in accordance with an embodiment of the present invention.

FIG. 6A is a diagram of a representative graphical user interface in which the user can select socio-economic categories and health outcomes in accordance with an embodiment of the invention.

FIG. 6B is a diagram of a representative graphical user interface in which the user is presented with alternative socio-economic models and predictiveness measures.

### DETAILED DESCRIPTION OF EMBODIMENTS

Disclosed herein are certain embodiments of a socio-economic score and indexing system 10 that visually presents a user an adjustable socio-economic algorithm for generating socio-economic models. These models can be subsequently used for indexing patients or patient populations as outlined in detail below.

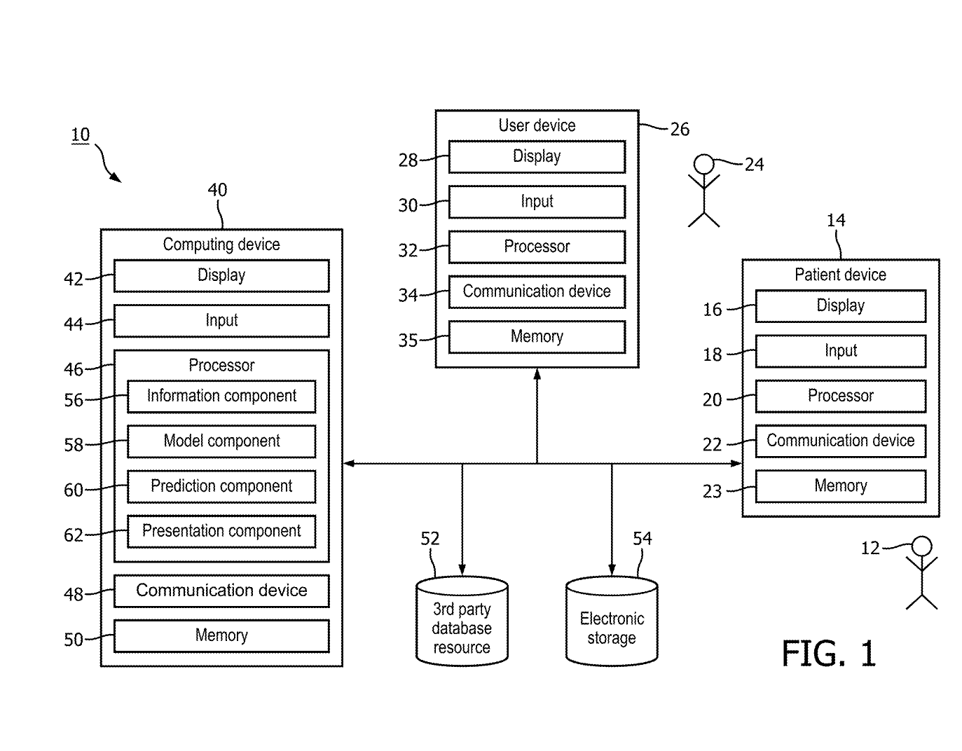

The socio-economic system 10 provides a measure of a variety of socio-economic factors, which have been found to be equally, if not more important, to predicting clinical outcomes than clinical factors. With reference to FIG. 1, the system 10 may be accessed by an individual 12 such as a patient who interacts with the system 10 via a patient device 14. The patient device 14 may be any of a plurality of devices with computing capability such as a desktop computer, portable computer, tablet or smartphone. The patient device 14 includes a display 16, an input 18, a processor 20, a communications device 22, and memory 23. As is well-known in the art, the display 16 may present a graphical user interface with menus, tables, icons, and the like to permit interaction with the system 10. The input 18 may be any of a variety of input devices such as a keyboard, touchscreen, computer mouse, trackball, voice recognition and the like capable of allowing the individual 12 to interact with the graphical user interface presented on the display 16. The communication device 22 is any communication device capable of connecting the patient device 14 to the system 10 such as Ethernet, Wifi, Bluetooth, GSM, CDMA or the like used alone or in combination to facilitate communications. The memory 23 may be volatile, non-volatile or both. Among other things, the patient device 14 may be used by the patient 12 to communicate self-reported health status. For instance, the patient may be asked to report whether they feel bad, good, or excellent. Alternatively, the patient may be able to report their health status on a given scale with numeric values between 0-10. Of course, one of ordinary skill in the art can appreciate that a variety of self-reporting schemes can be used with a variety of scales without departing from the spirit of the presentation invention.

Similarly, a user 24 may be physician, nurse, administrator, or other caregiver. The user 24 may access the system 10 via a user device 26. As with the patient device 14, user device 26 also includes a display 28, input 30, processor 32, a communications device 34, and a memory 35. As is well-known in the art, the display 28 may present a graphical user interface with menus, tables, icons, and the like to permit interaction with the system. The input 30 may be any of a variety of input devices such as a keyboard, touchscreen, computer mouse, trackball, voice recognition, and the like capable of allowing the user to interact with the graphical user interface presented on the display 28. The communications device 34 may be any communication device capable of connecting the user device 26 to the system 10 such as Ethernet, Wi-Fi, Bluetooth, GSM, CDMA, or the like.

The system also includes a computing device, or computer, 40 that also includes a display 42, input 44, processor 46, a communications device 48, and memory 50. As is well-known in the art, the display 42 may present a graphical user interface with menus, tables, icons, and the like to permit interaction with the system 10. The input 44 may be any of a variety of input devices such as a keyboard, touchscreen, computer mouse, trackball, voice recognition, and the like capable of allowing the user to interact with the graphical user interface presented on the display 42. The communication device 48 may be any communication device capable of connecting the computer 40 to the system 10 such as Ethernet, WiFi Bluetooth, GSM, CDMA, or the like.

The system 10 also includes access to third party database resources 52 which may be located on-site or accessed remotely from third party providers. For instance, clinical information regarding the individuals 12 may be retrieved from an electronic medical records (EMR) database from providers such as Epic, Inc. or Cerner, Inc. In addition, socio-economic information may be retrieved from a socio-economic information databases from providers such as CentraForce, Inc. The system may also include electronic storage 54 which may include private information regarding individuals collected by a healthcare provider. The database resource 52 and electronic storage 54 may be co-located within the first user device 14, the second user device 26, the computer or remote (as depicted). Ideally, given the size of the databases contained in either the database resources or electronic storage, these databases 52, 54 are preferably contained in cloud-based data storage such as that provided by SalesForce.com, Inc, Amazon Web Services, Inc. or other similar providers.

The processor 46 of computing device 40 includes an individual component 56, a model component 58, a prediction component 60, and a presentation component 62. The individual component 56 is configured to process information about individuals 12 such as claims data, clinical data or socio economic data received from the databases 52, 54. The model component 58 is configured to define and train a socio-economic model, which correlates various socio-economic factors as explanatory variables with clinical outcomes, costs or self-reported patient health status as response variables via weighting factors associated with each factor. Lastly, the presentation component 62 displays the output of the model to the user 24 on display 16 and inquires whether the user 24 wishes to accept the model or make adjustments and retrain the model. The user provides these responses via the input 18.

While the network environment of the system depicted in FIG. 1 contemplates that the patient device 14, the user device 26 and the computing device 40 may be remote from one another, one of ordinary skill in the art can readily appreciate that one or more of these components could be co-located in a single device or that further segregation of components could be made without departing from the scope of the present invention.

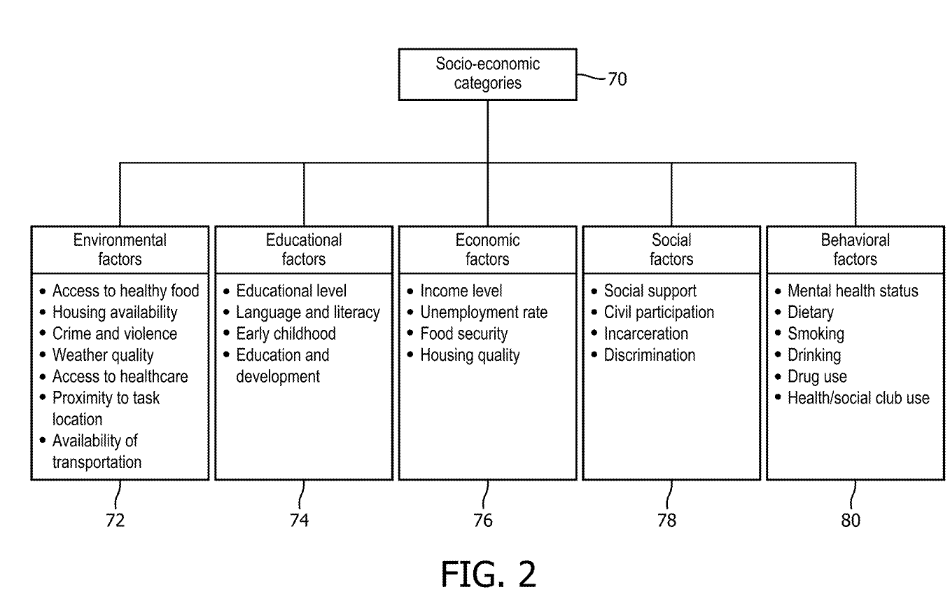

As seen in FIG. 2, there are multiple socio-economic factors that can be taken into account. FIG. 2 provides a non-exhaustive example of some of the more common socio-economic categories and factors. Here, the socio-economic categories 70 are represented as 5 categories: Environmental 72, Educational 74, Economic 76, Social 78, and Behavioral 80 categories. In turn, each of these categories 72-80 may include various related factors. For instance, as depicted, the environmental category 72 includes factors such as access to healthy food, housing availability, crime and violence rate, and weather quality. The education category 74 includes factors such as education level, language and literacy, and early childhood development. The economic category 76 includes factors such as income level, unemployment rate, food scarcity, housing quality. The social category 78 includes factors such as social support network, civil participation, incarceration, and discrimination. Lastly, the behavioral category 80 includes factors such as smoking habit, drinking, drug use, dietary status, health/social club use, and mental health status. While not exhaustive, this list provides a representative example of the socio-economic factors that are generally seen as impacting an individual's health and clinical outcome. Beyond the clinical information captured in an EMR or readily ascertainable during a consultation, it has been found that these features have a significant impact the individual's health and likely outcome. Currently, caregivers have little insight into these factors and are thus unable to consider the entire picture when seeking to treat a patient often leading to costly sub-optimal outcomes.

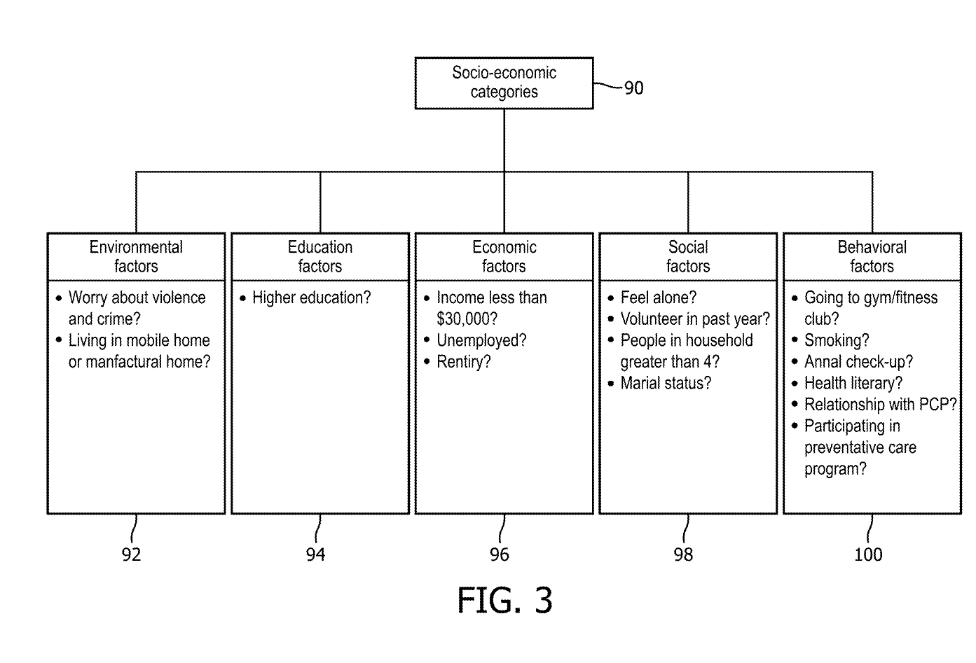

While having access to all of the information depicted on FIG. 1 would be ideal, much of this data has proven difficult to collect and process. As such, simplifications can made to focus on key factors for which sufficient data exists. One embodiment, which seeks to simplify the factors utilized, is depicted in FIG. 3. Here, the socio-economic categories 90 have been simplified to include environmental 92, educational 94, economic 96, social 98, and behavioral 100 factors. In this simplified embodiment, the environmental category 92 includes two factors: does the individual worry about crime and violence, and are you living in a mobile home, manufactured home or other temporary structure. As for the educational category, the only factor is whether the individual has attained higher education or not. The economic category 96 includes factors such as whether the individual's income level is higher or lower than a threshold (e.g. $30,000), whether the individual is unemployed or not, and the individual has stable housing or not (e.g. renting versus owning). The social category 98 includes factors such as whether the individual feels lonely, do they volunteer or participate in community events, the number of people currently living in their household, and marital status (e.g., married, single, divorced). Lastly, the behavioral category 100 includes factors such current gym participation, smoking, annual check-up, relationship with primary care physician, participation in preventative health activities. While less comprehensive than the factors listed in FIG. 1, these factors are more readily available and capable of being processed in a quick and efficient manner via data that is readily available. Of course, one of ordinary skill in the art can readily appreciate this example is merely demonstrative, a multitude of different factors could be added, deleted, or substituted without departing from the scope of the present invention.

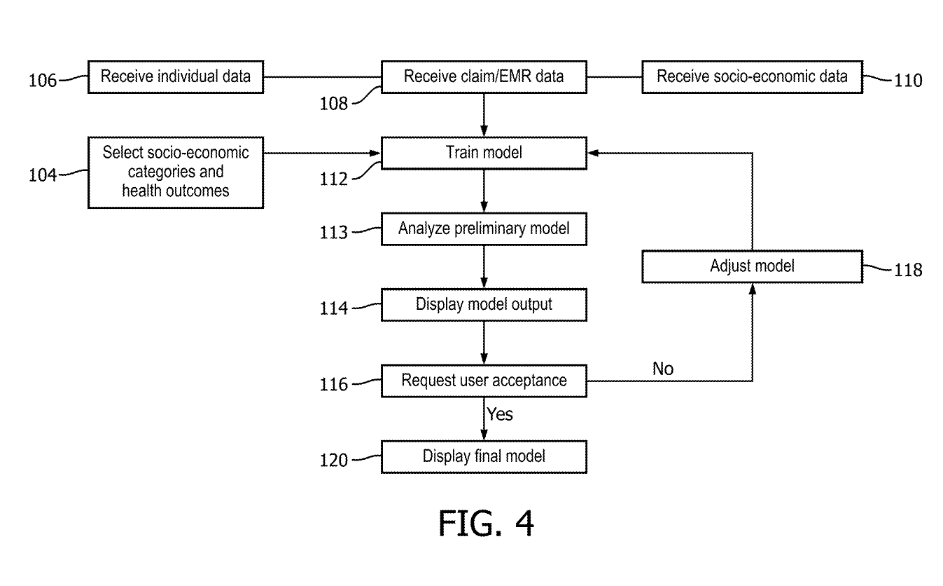

As seen in FIG. 4, the system 10 permits the user 24 to select which socio-economic categories 92-100 should be used in the model. The Individual Information component 56 receives individual data 106, claims or electronic medical records (EMR) data 108, and socio-economic data 110. The individual data 106 includes data received from the patient device 14. In a preferred embodiment, the user 24 will provide information regarding a self-assessment of their health status, which may be used as the response variable to train the model 122. Alternatively, the patient's 12 health status could be determined automatically by analyzing sensor-based output. As another option, the patient's 12 clinical medical records could be analyzed to provide an assessment of the individual's health status. As, will be described below, the patient's 12 health status is used as a response variable to train the model 122. For example, one would expect that an individual with low socio-economic factors, would report having a lower health status and vice versa. Aside from, self-reported health status, the inventors contemplate that the response variable of the model may be various other health outcomes models well known in the art such as the Charleston Comorbidity Index, A1C level, Total claim cost, Medication adherence, or self-reported health status reported by the patient 12. Of course, this list is not exhaustive and a variety of response variables could be used in the model.

The individual information component 56 also receives data from claims or EMR databases 108. Again, these databases may come from external providers stored on 3rd party database resources 52. Or, this data may be private in nature stored on electronic storage 54. In a preferred embodiment, the inventors contemplate that information regarding the patient's 12 age, ethnicity, marital status, and zip code or address can be extracted from the database 52, 54. This data can be used as inputs to extract the appropriate data from the socio-economic database contained on a third party database resource 52, or electronic storage 54.

The individual information component 56 also receives data from socio-economic databases 110, which is extracted based on the EMR and/or claims data received from claims or EMR databases 108. The socio-economic data 110 may be located on external data resources 52 such as from a public provider like CentraForce. Alternatively, the socio-economic data 104 may be a private database stored in electronic storage 54, which may be resident with computing device 40, or remote from computing device 40.

In step 104, the computing device 40 uses the data 106, 108, and 110 to select the relevant data related to the selected factors 92-100 of the categories 90. For example, the system will use the individual's zipcode or address to extract the relevant socio-economic factors associated with the patient's zipcode or address while excluding the factors that are not as predictive based on the desired outcome model. Moreover, if the user 24 desires to build a model related to A1C levels to determine a patient's risk of getting diabetes while dietary and health club use may be highly predictive, mental health may be less predictive and thus the system could be configured to exclude less predictive features. Next, in step 112, the system trains a model 122 using the selected data to provide a socio-economic score 122. Machine learning techniques are applied to create a model 122, which fits the data and is predictive. The model includes socio-economic factors 134 and an associated weighting factor 136. For example, the computing device 40 may calculate a model 122 such as:

SES=(Af)*(Age)+(Uf)*Unemployment+(AQf)*Air quality, where
SES is the Socio-economic Score 126;

Age is the age of the patient 134;

Unemployment represents the unemployment status of the patient 134;

Air quality represents the air quality the patient is exposed to 134

Af is the Age weighting factor 136;

Uf is the unemployment factor 136; and

AQf is the Air Quality factor 136.

The inventors contemplate that in a preferred embodiment linear regression may be employed to develop the socio-economic model 122. However, a variety of other techniques can be employed as well such as Bayesian linear regression, least-angle regression, theil-sen estimator, Lasso, Ridge, or even polynomial regression techniques could be employed without deviating from the scope of the present invention.

In step 113, the prediction component 60 calculates the predictive quality of the model 122 generated by the model component 58. In a preferred embodiment this can be accomplished by performing a Root-Mean-Square Error (RMSE) and R-Square R² values for the model 122. These values are well-known in the art and would provide the user with a ready metric indicating the quality of the model 122. Of course, a variety of other techniques could be used to assess the predictive quality of the model 122 such as average mean variance, median mean variance, and average absolute deviation.

The presentation component 62 displays the model generated by the model component 58 and the predictive quality of the model generated by the prediction component 60 for the user 24 on display 28 in step 114. In step 116, the user 24 is given the option to accept or reject the model 122. If the user 24 accepts the model, the user 24 is given the option to display and save the model 120. If the user rejects the model, the user is allowed to adjust the model in step 118 via input 30 of the user device 26.

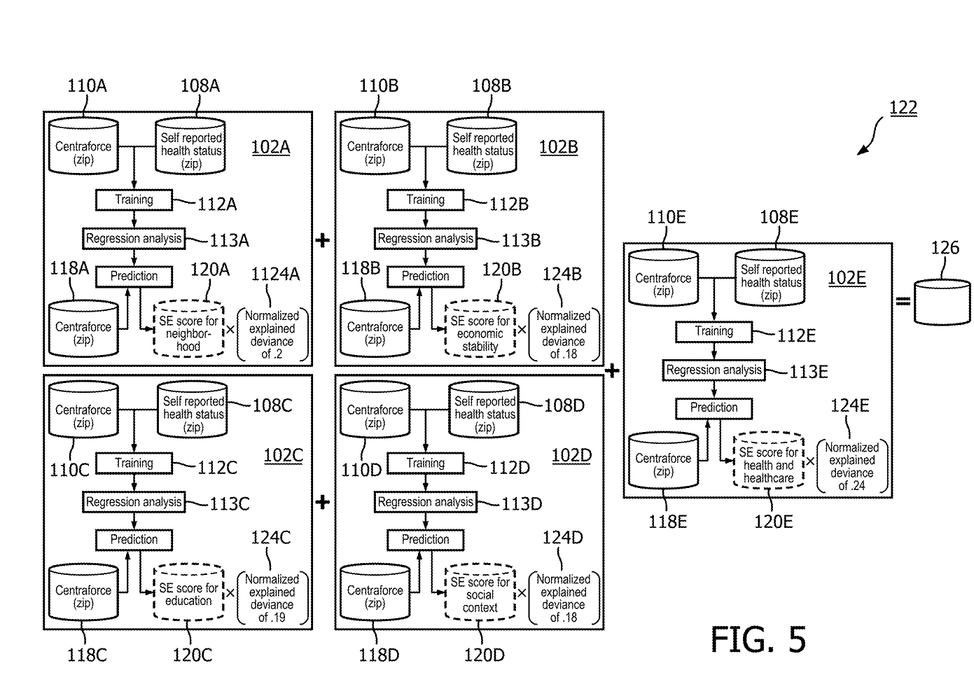

While the preceding description is one demonstrative embodiment of the invention wherein the model is generated with each socio-economic category simultaneously to generate a singular socio-economic model, the inventors contemplate, as seen in FIG. 5, that the it may be advantageous to generate the socio-economic model 122 based on individual socio-economic models for each category 92-100. This allows for each category to be individually calculated and weighted. The weighted categories can then be combined into a singular socio-economic model. FIG. 5 shows an embodiment of this configuration of the invention wherein like features are given the same reference as described above with respect to FIG. 4 wherein the Environment category references are appended with the letter A, the Economic category are appended with the letter B, the Educational category references are appended with the letter C, the Social category references are appended with the letter D, and the Behavioral category references are appended with the letter E. Each category 102A-102E includes a secondary weighting component 124A-124E. The results are then summed to create the socio-economic model. 122.

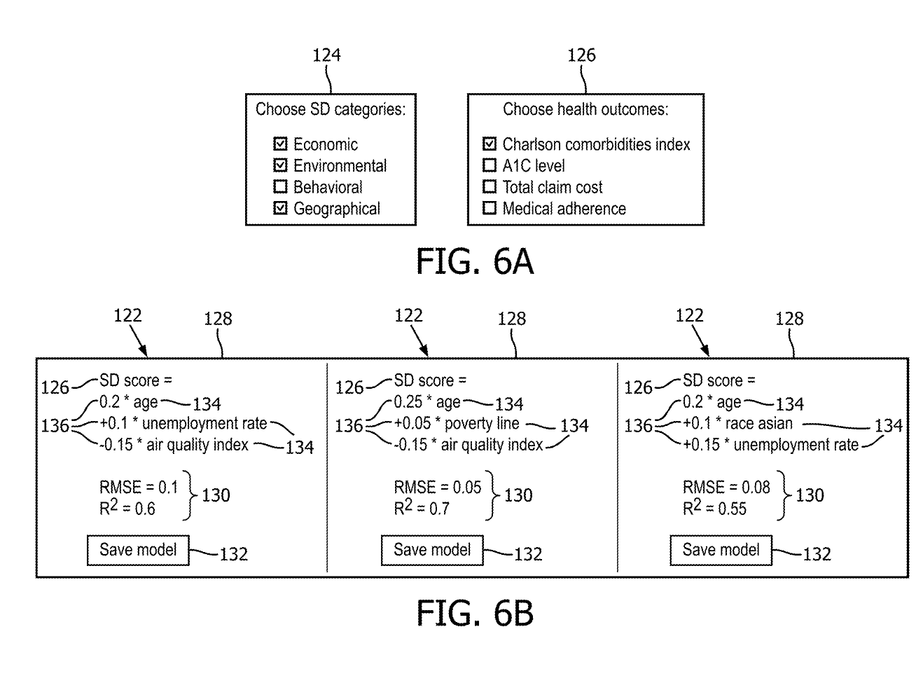

Another unique feature of the present invention is its customizability, which allows users to customize the model 122 as desired to fit their particular application. With reference to FIG. 6A, a demonstrative graphical user interface is shown which would be presented on the display 28 of the user device 26. Here, the user 24 can select which socio-economic categories should be included when generating the socio-economic model 122. For instance in the example shown, the Economic 96, Environmental 92, and Social 98 categories have been selected by the user 24 represented by the checks in the displayed boxes. For this model 122, the user has concluded that the behavioral category is not relevant to the model. This could be for experimental purposes or it could be that the user 24 has concluded that behavioral factors are irrelevant, or even worse, may detune the model 122 and decreased predictiveness.

FIG. 6A further shows that the system 10 provides a user interface that allows the user 24 to select the socio-economic categories 124 and to select the desired health outcome metric or socio-economic score 126. Once the system has generated the socio-economic model, it can be used in a variety of manners. For instance, the output can be used in isolation with respect to a particular patient 12 to calculate a numeric index representing an assessment of their socio-economic status. For instance, conditions may exist to characterize certain values as representing high risk (e.g., index in top ⅓ of patient population), medium risk (e.g., index in middle ⅓ of patient population), or low risk (e.g., index in bottom ⅓ of patient population). Alternatively, the output could be a plurality of indices representing the socio-economic index of a plurality of patents 12 for comparative analysis. This would allow a user 24 to rank risk and triage, or prioritize, patient's 12 based on risk. The user 24 could determine that the highest at risk individuals will require additional socio-economic support (e.g., subsidies, at-home nursing, transportation, emotional support, etc.) as compared to individuals with low socio-economic risk scores, thus optimizing care.

Alternatively, the inventors contemplate that the socio-economic score can also be used to show the correlation of socio-economic factors to various health outcomes such as the Charleston Comorbidity Index, A1C level, Total claim cost, Medication adherence. As seen I FIG. 6A, the user 24 may select the health outcome or socio-economic score 126 desired, which will be used as the response variable in the socio-economic model 122. In a further embodiment, the model 122 could be used to adjust the selected health outcomes model 122 to create a new composite metric, which includes both socio-economic and clinical factors. This new metric can be used to provide comprehensive representation of the patient's health and likely outcome based on both clinical and socio-economic factors. This may be accomplished via a variety of means. In one embodiment, the health outcome metric may be scaled by multiplying the calculated health outcome with the socio-economic index calculated by the computing device 40 for a particular patient 24 and a scaling factor could be applied to relate these two variable and provide a scaled health outcomes metric which accounts for both clinical factors and socio-economic factors. For instance, in one embodiment, the scaled health outcome may be calculated as follows:

SHO=HO*SEI*Sf, where

SHO is the scaled health outcome;

HO is the health outcome;

SEI is the socio-economic index for a particular patient; and

Sf is a scaling factor to relate the health outcome with the Socio-economic Index.

FIG. 6B shows a representative user interface presented on display 28 with the model and quality metrics displayed of step 114. For each of the selected categories 92-100, the adjustable model 122 is displayed showing the factors 134 including their associated weighting factors 136. In addition, this depicted graphical user interface also provides exemplary RMSE and R² values 130 to provide the user 24 with an estimate of the model's 122 predictive quality. The user 24 can accept each of these models as final and press the save button 132, or the user 124 can make adjustments to the adjustable model 128 in step 118 and resubmit the model to the model component 58 to recalculate a new version of the model in step 112.

In step 104, the display 28 provides the user 24 with the socio-economic categories 92-100 and health outcomes 126 from which the user 24 may choose. This uniquely provides the user 24 with the flexibility to determine which socio-economic categories 92-100 should be included in the model 122 and which categories 92-100 the user 24 wishes the system 10 to exclude from the analysis. Next the user selects the desired health outcome 126. The model component of the computing device will select the appropriate socio-economic factors based on the selected socio-economic categories 124 and the selected health outcomes 126. Given that the system 10 also calculates predictiveness metrics in step 113. The user 24 can generate multiple models 122 using different categories 92-100 and evaluate which ones provide the best correlation.

The present invention can be used for a variety of advantageous purposes. For instance, as noted above, physicians are increasingly being compensated, or incentivized, based on efficiency. However, physicians identified as low performers often complain that their patient population is sicker or more complicated than the average and rather than incentivizing efficiency such systems encourage doctors to abandon complicated, or costly patients 12 while doctors who take on complicated patients 12 are penalized. Accordingly, providing administrators, payors, or governmental organizations a reliable tool that can benchmark a provider's patient population would go far to address this problem in the art, which could lead to a fairer compensation schemes which account for socio-economic factors. This in turn might encourage physicians to desire complicated patients since they would represent the greatest opportunity for therapeutic improvement and compensation, if successful.

The present invention can also be used to enhance care if provided to a physician, nurse or other caregiver. The user 24 may be able to have a quick assessment regarding socio-economic factors that may complicate care, and as noted above, may be more important to determining patient's health outcome than clinical data. Typically, physicians had little insight into these components, but simply providing the caregiver with all the data on a patient may not solve the problem even if the patient would be willing to provide it. Most caregivers are under extreme time pressures and are not data scientists. Finding this data, analyzing it and determining the correlations with the health outcomes of interest is simply too time consuming and complicated to be performed by a caregiver in real time. The present invention could provide caregivers with a ready tool to provide quick index regarding a patient's socio-economic factors that may impact outcomes so they can focus additional services on the patients that are most likely in need of additional support and avoid providing services to patients that don't need additional support. In addition, if the socio-economic score is high to a patient due to behavioral factors. The user may, as part of their care plan, prescribe social services such as a counselling, a support group, etc. Or, if the patient is being discharged, the discharging physician may consider whether additional community support or at-home care is justified in an effort to avoid the cost associated with readmission. So, in a way, the present invention could provide socio-economic decision support mirroring clinical decision support which is ubiquitous in the industry.

As another example, administrators have extreme pressures put on them by payers to contain costs as healthcare organizations continue to consolidate, it becomes increasingly difficult for administrators to determine where they have inefficiencies in their large and growing organization. The present invention can help them analyze the actual impact their providers are having on their patients and highlight areas where improvements can have the greatest impact on patient outcomes and reduce costs.

Although the description identifies or describes specifics of one or more embodiments, such specifics are not necessarily part of every embodiment, nor are all various stated advantages associated with a single embodiment. On the contrary, the intent is to cover all alternatives, modifications and equivalents included within the scope of the socio-economic system and method as defined by the appended claims. Further, it should be appreciated in the context of the present disclosure that the claims are not necessarily limited to the particular embodiments set out in the description.

Note that reference to thresholds refers to minimum triggers for certain conditions for actions to commence. The thresholds may be based on historical or experimental data, or based on the expertise of a user and/or context. In some embodiments, a threshold may be established based on a combination of experience and context.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Any reference signs in the claims should be not construed as limiting the scope.

At least the following is claimed:

1. A system for displaying adjustable socio-economic model, comprising:
a computing device;
a database containing socio-economic data, each socio-economic category includes one or socio-economic factors; and
at least one user device with a display, and an input, wherein the user device is configured to render a graphical user interface on the display, the graphical user interface displaying socio-economic categories, wherein the user may select one or more socio-economic category, wherein the one or more selected socio-economic category is communicated to the computing device to train a socio-economic model that correlates the one or more socio-economic factors for each socio-economic category with a socio-economic score, wherein the model includes a plurality of socio-economic factors and a plurality of weighting factors, the computing device also calculates one or more predictiveness metrics which indicates the quality of the socio-economic model calculated by the computing device to correlate the socio-economic factors with the selected socio-economic score, the computing device communicates the socio-economic model to graphical user interface on the user device, and the computing device communicates the one or more predictiveness metrics to the graphical user interface on the user device.

2. The system of claim 1, wherein the socio-economic categories comprise an environmental category, an educational category, an economic category, a social category, and a behavior category.

3. The system of claim 1, wherein the health outcomes comprise a Charleston Comorbidities Index, A1c Levels, Total Claim Cost, Medication Adherence, or patient self-reported health status.

4. The system of claim 1, wherein the socio-economic model trained by the computing device comprises a sub-model trained by the computing device for each socio-economic category, the computing device then multiplies each sub-model with a second weighting factor and then sums the weighted sub-models to create the socio-economic model.

5. The system as recited in claim 1, wherein the socio-economic model displayed on the user device is adjustable, wherein the user may adjust the weighting factors of the socio-economic model and resubmit the model to the computing to train an updated socio-economic model and display updated predictiveness metrics.

6. The system of claim 1, wherein the computing device calculates a numeric index for an individual by imputing the socio-economic data for the individual.

7. The system of claim 1, wherein the computing device calculates a numeric index for a plurality of individuals within a selected patient population, wherein socio-economic data for each individual is input into the socio-economic model to generate the numeric index which represents the socio-economic risk of each individual.

8. The system of claim 6, wherein the graphic user interface further displays a plurality of health outcomes, wherein the user may select a health outcome, and wherein the computing device uses the socio-economic index to scale the health outcome score by multiplying the health outcome score with the socio-economic index and a scaling factor.

9. A computer-implemented socio-economic indexing system, comprising:
receiving individual data, electronic medical records data, and socio-economic data;
displaying socio-economic categories on a graphical user interface of a user device such that the user may select one or more socio-economic categories from the socio-economic categories displayed on the graphical user interface;
training a socio-economic model which correlates the selected socio-economic categories with a socio-economic score, wherein the socio-economic model includes a plurality of socio-economic factors and a plurality of weighting factors; and
rendering a graphical user interface on the user device to display the socio-economic model and one or more predictiveness metrics which indicate the quality of the socio-economic model to correlate the socio-economic categories with the selected socio-economic score.

10. The method as recited in claim 9, wherein the socio-economic categories comprise an environmental category, an educational category, an economic category, a social category, and a behavior category.

11. The method as recited in claim 9, wherein the displaying step further includes displaying one or more health outcomes on the graphical user interface of the user device such that the user may select a health outcome, and wherein the method further includes calculating a health outcome index for an individual.

12. The method of claim 11, wherein the method further calculates socio-economic index for an individual using the received data to generate a socio-economic index for the individual.

13. The method of claim 12, wherein the method further comprises calculating a scaled health outcome index, wherein the wherein the scaled health outcome index is calculated by multiplying the health outcome index with the socio-economic index and a scaling factor.

14. The method of claim 9, wherein the socio-economic model displayed in the rendering step is adjustable such that the user may adjust the weighting factors and resubmit the socio-economic model to the training step to train an updated socio-economic model and an updated predictiveness metric.

15. A non-transitory computer readable medium encoded with executable instructions that, when executed by one or more processors, causes the one or more processors to:
receive individual data, electronic medical records data, and socio-economic data;
display socio-economic categories on a graphical user interface of a user device such that the user may select one or more socio-economic categories from the socio-economic categories displayed on the graphical user interface;
train a socio-economic model which correlates the selected socio-economic categories with a socio-economic score, wherein the socio-economic model includes a plurality of socio-economic factors and a plurality of weighting factors; and
render a graphical user interface on the user device to display the socio-economic model and one or more predictiveness metrics which indicate the quality of the socio-economic model to correlate the socio-economic categories with the selected socio-economic score.

16. The non-transitory computer readable medium as recited in claim 15, wherein the socio-economic categories comprise an environmental category, an educational category, an economic category, a social category, and a behavior category.

17. The non-transitory computer readable medium as recited in claim 15, wherein the socio-economic model displayed in the rendering step is adjustable such that the user may adjust the weighting factors and resubmit the socio-economic model to the training step to train an updated socio-economic model and an updated predictiveness metric.

18. The non-transitory computer readable medium of claim 15, wherein the displaying step further includes displaying one or more health outcomes on the graphical user interface of the user device such that the user may select a health outcome, and wherein the method further includes calculating a health outcome index.

19. The non-transitory computer readable medium of claim 18, wherein the one or more processors calculate a socio-economic index for an individual using the received data to generate a socio-economic index for the individual.

20. The non-transitory computer readable medium of claim 19, wherein the one or more processors further calculating a scaled health outcome index, the wherein the scaled health outcome is calculated by multiplying the health outcome index with the socio-economic index and a scaling factor.
